(19) United States
(12) Patent Application Publication
(10) Pub. No.: US 2013/0344598 A1
(43) Pub. Date: Dec. 26, 2013

(54) NEURAL CELL PURIFICATION FOR TRANSPLANTATION

(71) Applicant: Gabriel Nistor, Laguna Niguel, CA (US)

(72) Inventor: Gabriel Nistor, Laguna Niguel, CA (US)

(73) Assignee: California Stem Cell, Inc., Irvine, CA (US)

(21) Appl. No.: 13/981,020

(22) PCT Filed: Jan. 24, 2012
(86) PCT No.: PCT/US12/22389

§ 371 Date: Sep. 13, 2013

Related U.S. Application Data
(60) Provisional application No. 61/461,888, filed on Jan. 24, 2011.

(51) Int. Cl. C12N 5/0793

(52) U.S. Cl. CPC C12N 5/0619
USPC 435/368; 435/402; 435/305.2

(57) ABSTRACT

Aspects of implementations wherein reductions in contamination in transplantation cell population are disclosed herein, including incubating a suspended population of live cells in a culture medium with a substrate having surface that promotes cell to cell adhesion. During the process, the incubating is in absence of substantial mechanical disturbance of the culture medium and cells are in the culture medium for a predetermined period of time. A resulting population of cells which is relatively reduced or eliminated in contaminants is taught. Said population of cells may be collectable by centrifugation and is separable from said contaminants by centrifugation.

### FIELD

This disclosure relates to processes, devices, systems and methods for purifying neural cells.

### BACKGROUND

As an important step in preparation for transplantation, cell cultures typically undergo some form of dissociation. Neurons cultured in-vitro are especially sensitive to enzymatic dissociation or mechanical manipulations. Following such manipulation, immediate viability is 75% to 90%. More cell death occurs after a few hours following dissociation, resulting in a cumulated viability of 60-80%. Therefore, it is expected that transplantation of the dissociated population will include 10% to 40% nonviable cells, resulting in a “cleaning” mechanism from the host.

Living cells express cellular adhesion molecules (CAM) on the cell surface, which allow them to attach to various substrates or have intercellular interactions. There are four classes of CAM proteins: Ig (immunoglobulin) superfamily (IgSF CAMs), integrins, cadherins, and selectins. The IgSF-CAMs are calcium independent and homophilic or heterophilic. The family is represented by various molecules: SynCAMs (Synaptic Cell Adhesion Molecules), NCAMs (Neural Cell Adhesion Molecules), ICAM-1 (Intercellular Cell Adhesion Molecule), VCAM-1 (Vascular Cell Adhesion Molecule), PECAM-1 (Platelet-endothelial Cell Adhesion Molecule), L1, CHL1, MAG, nectins and nectin-like molecules. The cadherins are homophilic molecules, calcium dependent, mostly represented by E-cadherins (epithelial), P-cadherins (placental), and N-cadherins (neural).

In general, homophilic binding occurs between cis- and trans-arrangements of the same molecule and allows for attachment/aggregation of the same type of cell, while heterophilic molecules allows for attachment to various substrates. CAMs play important roles in cell biology, cell migration, proliferation, and differentiation.

Undifferentiated mouse stem cells may have relatively high quantities of ICAM-1, VCAM-1, and NCAM antigens on their surface. However, the specific receptors of these cellular adhesion molecules, namely, Mac-1, LFA-1, and VLA-4, were not detected. Moreover, 12 hours after withdrawal of Leukemia Inhibitory Factor (LIF), these molecules were not detectable (Tian et al, 1997). The presence of the mentioned CAMs seems to be related to the use of LIF in the culture methods of mouse embryonic stem cells. It is not believed to be translated to of the human embryonic stem cells. NCAMs and N-cadherins are abundant in the nervous system and cause neurite growth, CNS development, maturation and plasticity. These adhesion molecules contribute to formation of neurospheres.

Neurospheres are spherical structures of cells resulting from clonal expansion of individual neural progenitors in the presence of a mitogen (Reynolds et al, 1992). Cell to sphere aggregation, or sphere to sphere aggregation, may play a role in neurosphere formation (Mori et al, 2006, 2007; Singec et al, 2006). Neurosphere technology is used to promote neural progenitor cell proliferation. Similar sphere forming assays are used to evaluate skin cells, breast, pancreas, muscle or cancer cells.

In some exemplary implementations the process, system, device or method herein discloses the purification of neural cells by elimination of nonviable cells. In some instances by eliminating a portion of the nonviable cells the overall viability of a transplant population can reach at least 75%, at least 80%, at least 85%, at least 90%, at least 95%, at least 99%, about 75%, about 80%, about 85%, about 90%, about 95%, about 99%, or about 100° A. What is also encompassed is the above parameters that are at least about above 95%, and at least about above 99%.

In some exemplary implementations the process, system, device or method herein discloses the purification of neural cells by elimination of viable cells outside of the neural lineage. In some instances by eliminating a portion of the viable non neural lineage cells the overall viability of a transplant population can reach at least 75%, at least 80%, at least 85%, at least 90%, at least 95%, at least 99%, above 75%, above 80%, above 85%, above 90%, above 95%, about 99%, or about 100%, and the like. What is also encompassed is the above parameters that are at least about above 95%, and at least about above 99%.

In some exemplary implementations the process, system, device or method herein discloses the purification of neural cells by elimination of cell types that are not of the neural lineage, such as undifferentiated stem cells. In some instances by eliminating a portion of the undifferentiated stem cells the overall viability of a transplant population can reach at least above about 95%. In some instances by eliminating a portion of the overall viability of a transplant population can reach at least above about 99%. In some instances by eliminating a portion of the undifferentiated stem cells the overall viability of a transplant population can reach above 99%.

In other exemplary implementations the process, system, device, and method herein discloses transferring enzyme-dissociated cells on a low adherent substrate and allowing for cellular aggregation to occur for a defined amount of time in the absence of exogenous adhesion promoting molecules, such as fibronectin, laminin, and the like. In some instances the low adherent substrate can be a highly hydrophobic material, for example, polyethylene, or untreated polystyrene. In some instances coating is performed to achieve the low adherent substrate. Coatings can be used on regular plastic culture containers to achieve the low cell adherence, for example agar-agar or derivatives. Low adherent surfaces and ultra-low adherent surfaces are surfaces that promote cell to cell adhesion.

In some exemplary implementations the process, system, device, or method herein discloses that time control is useful to control or influence the size of the aggregates (microspheres). In some instances by allowing more time for the cell- to-cell interactions, small aggregates may fuse and form larger aggregates. In some instances uniform and regular size of aggregates can be achieved by placing the suspension of cells in an array which may contain micro-wells or the like. In some instances control of vibration and disturbance of the resting cell suspension can be used to achieve the homogeneous distribution and size of the microspheres.

In some exemplary implementations a system and device provides consistent size and composition of one or more microspheres. The device can have a substantially flat bottom container. In some instance it may have shallow extruded wells preferred 0.01 to 0.1 mm which may also be polygonal, such as pentagonal, hexagonal shape (honeycomb pattern), heptagonal, octagonal, and the like. Coating the inner surface of said container is a low adherent surface. In some instances said low adherent surface comprises at least one of polyethylene and fluoropolymers. Without implying any limitation, the extruded well can have a diameter or a depth of about 0.01 mm, about 0.02 mm, about 0.05 mm, about 0.1 mm, about 0.2 mm, about 0.5 mm, about 1.0 mm, about 2.0 mm, and so on.

In some exemplary implementations, the present disclosure provides a system and device to provide consistent size and composition of microspheres. The device having a substantially flat bottom container. In some instance it may have shallow extruded wells preferred 0.01 to 0.1 mm which may also be hexagonal shape (honeycomb pattern). Coating the inner surface of said container is a low adherent substrate. In some instances said low adherent surface is at least one of polyethylene and fluoropolymers. In some instances the low adherent substrate is applied by extruding, embossing, stamping, etching or film application. In some instance the application of substrate is by a mold or reusable mold which for stamping. In some instances the application of substrate is via mask(s) or reusable mask(s) for a photo-resist/etching procedure.

### SUMMARY OF THE INVENTION

The present disclosure provides method to reduce contamination in a cell population, the method comprising: a. incubating a suspended population of live cells in a culture medium, b. with a substrate having surface that promotes cell to cell adhesion, c. wherein the incubating is in absence of mechanical disturbance of the culture medium, d. wherein cells are incubated in the culture medium for a predetermined period of time, e. resulting in a population of cells that is relatively reduced or eliminated in contaminants, f. wherein the population of cells that is relatively reduced or eliminated in contaminants is collectable by centrifugation and is separable from said contaminants by centrifugation. In another aspect, what is provided is the above method, wherein the substrate is a low adherent coating, the above method wherein the substrate is a low adherent coating that comprises one or more of a highly hydrophobic material, fluoropolymer, polyethylene, or polystyrene, the above method wherein containments which are reduced or eliminated are undifferentiated stem cells, the above method wherein contaminates which are reduced or eliminated are non-viable cells, the above method, wherein the substrate has surface properties that encourage or enhance cell to cell adhesion, the above method wherein the population of live cells are neural cells, the above method, wherein the population of live cells are neuronal progenitors, the above method, wherein the substrate has a bottom that is substantially flat, wherein the flat bottom minimizes cell agglomerates, as compared to a substrate that comprises a round bottom well, the above method, wherein the population of live cells which are incubated or interacted with the substrate are seeded at a density of about 100,000 to about 200,000 per cm², the above method, wherein the predetermined time is between about 3 and about 24 hours, the above method, wherein the method further comprising keeping the cell suspension generally undisturbed by manipulations, in a substantially vibration free environment, as well as the above method, further comprising prior to seeding removing pre-existing cell agglomerates by a gravitational method.

What is also embraced, is the above method, wherein the medium contains microspheres, and the wherein the microspheres are collected by a gravitational method or by filtration, as well as the above method, wherein the gravitational method is centrifugation at low gravity force, and wherein the low gravity force is 80-150 rcf.

The present disclosure also embraces a cell population prepared by the above method, or combinations of the above methods, wherein the cell population is derived from human motor neuron progenitor cells, wherein at least 50% of the cells detectably express Tuj1, wherein fewer than 10% of the cells detectably express Oct-4, and wherein at least 50% of the cells reside in at least one microsphere. In another aspect, what is encompassed is the above cell population, wherein at least 80% of the cells detectably express Tuj1.

Furthermore, the present disclosure provides a device to form consistent size and composition microspheres comprising: a substantially flat bottom container; and, a low adherent substrate coated on the inner surface of said container. Additionally, the disclosure provides the above device, wherein said low adherent substrate comprises at least one of polystyrene, polyethylene, and fluoropolymers. Moreover, the disclosure provides the above device, wherein said low adherent substrate comprises a low adherent coating, as well as the above device obtained by at least one of extruding, embossing, stamping, etching and film, or wherein the device comprises an extrusion, that comprises an emboss, that comprises a stamp, that comprises an etching, or that comprises a film, and also the above device, wherein said low adherent substrate is applied by at least one of masks for a photo-resist or etching procedure. In another aspect, the invention encompasses the above device, wherein the container further comprises wells, or wherein the wells are between about 0.01 mm to about 0.1 mm deep, or wherein the wells are substantially polygonal in shape, or wherein the substantially polygonal wells are pentagonal, hexagonal, or octagonal in shape.

### BRIEF DESCRIPTIONS OF THE DRAWINGS

FIG. 1A: Unorganized agglomeration of cells in microsphere 100×. FIGS. 1B, 1C, and 1D, disclose neurospheres containing large number of cells 10× (B) with visible areas of organized structures—rosettes 20× (C) and 100× (D).

FIG. 2. Interactions of neural cells with neural cells (FIG. 2A), with dead cells (FIG. 2B), with non-neural cells (FIG. 2C), and picture of orientation of processes in microsphere (FIG. 2D).

FIG. 3. Microspheres after 12 hours of incubation, before wash (10×).

FIG. 4. Microspheres after wash and concentration (20×).

FIG. 5. Cell culture flask with flat bottom wells. B, C. Profile of a single well with different types of walls.

FIG. 6. Screen capture of the Excel spreadsheet used for calculations.

FIG. 7. Tuj1 expression following microsphere formation.

FIG. 8. Cells in the discarded material from microsphere formation in 0% hESC, 100% MNP group does not contain Oct-4.

FIG. 9. Laced cultures containing 6% hESC, 94% MNP after microsphere formation.

FIG. 10. Cells in the discarded material from microsphere formation in 6% hESC, 94% MNP group (Tuj1—light grey, Oct-4—lightest).

FIG. 11. Laced cultures containing 20% hESC, 80% MNP after microsphere formation.

FIG. 12. Cells in the discard from microsphere formation in 20% hESC, 80% MNP group.

FIG. 13. hESCs following 17 hours of suspension in low adherent flasks for ‘microsphere formation.’ (a) Low magnification (40×) imaging of cells reveals that spheres did not form. (b) Higher magnification (100×) imaging of cells reveals that hESCs mostly remained in single cells suspension, with few small clumps present.

FIG. 14. Accounting of number of hECSs before microsphere formation, number of hESCs undergoing microsphere formation, and number of viable cells.

### DETAILED DESCRIPTION

### Abbreviations

BSA. Bovine serum albumin.
CAM. Cellular adhesion molecules.
CTS. Cell transplant solution.
DAPI. 4′,6-diamidine-2′-phenylindole dihydrochloride.

### HBSS. Hanks' Balanced Salt Solution.

hESC. Human embryonic stem cells.
IC. Starting or initial population of cells.
MNP. Motor neuron progenitor cells.
PBS. Phosphate buffered saline.
PI. Propidium Iodide (a fluorescent label for DNA, used to identify non-viable cells).
RCF. Relative centrifugal force.
TD. Total discarded cells.
TM. Total initial number of cells.
TrpLE. Trypsin replacement.
TV or TVC. Total viable cells.

### Procedure(s) to Make Microspheres

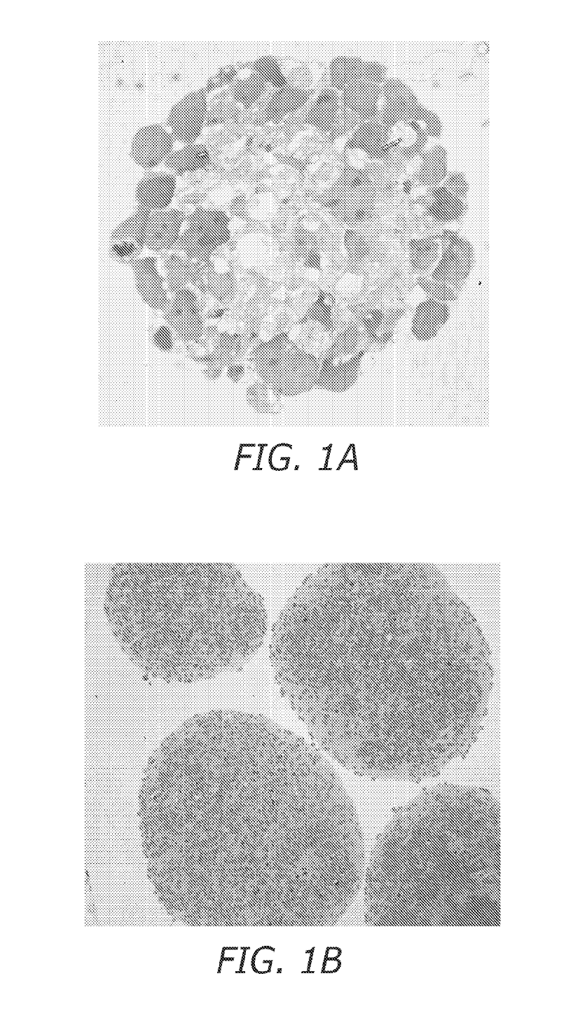

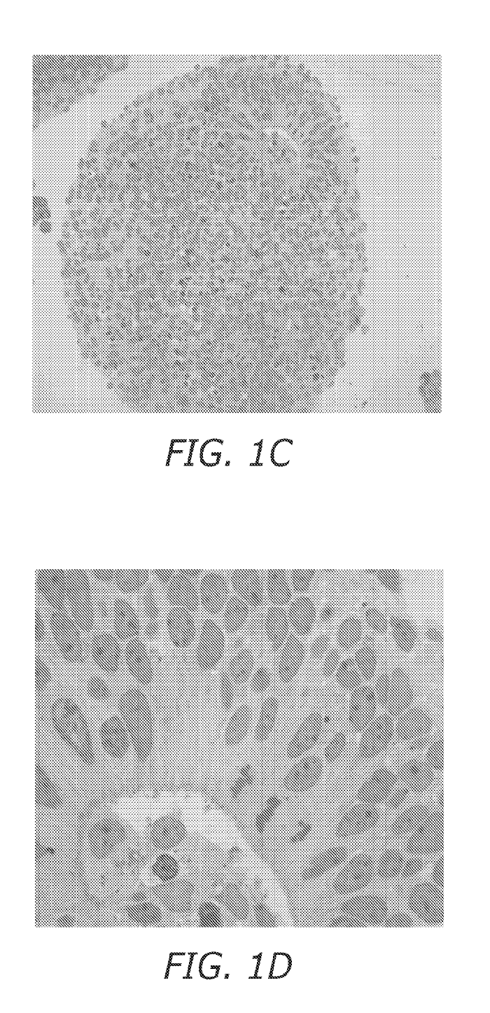

Neural microspheres are the exclusive result of aggregation of neural cells. When placed in a certain density and in absence of an adherent substrate, neural cells can extend short processes and eventually grab on each other based on cell expansions (Mori et al, 2007) and adhere to each other due to the homophilic properties of the surface CAMs. Neurospheres are the result of expansion of neural progenitors which leads to or requires a certain level of organization. Rosettes can be observed inside of the neurospheres, which are the direct result of cell multiplication. The difference between microspheres obtained by pure aggregation and a proliferative neurosphere forming process is disclosed in FIG. 1.

The size of the cell aggregates is directly controlled by the initial seeding density (Mori et al, 2007). Extended resting periods results in agglomeration and fusion of multiple microspheres and progressively increased size. The movement of the culture vessel or vibration can accelerate the agglomeration of the microspheres resulting in larger clumps by increasing the local density of the cells in some areas of the culture vessel. During the aggregation, no significant cell division occurs as demonstrated by the count of the total cells before and after microsphere formation. In contrast to previous work (Raynolds, 1992; Mori 2006), the present disclosure of microsphere formation does not utilize mitogens during aggregation and the scope is different from neural progenitor expansion. In addition, this process of microsphere formation is used on a population of cells that is predominantly adult cells, or that is predominantly post-mitotic neurons.

In exemplary embodiments, the cell population comprises at least 20% adult cells, at least 30%, at least 40%, at least 50% at least 60%, at least 70%, at least 80%, at least 90%, at least 95%, at least 98%, and the like, adult cells. In other aspects, the cell population comprises at least 20% post-mitotic neurons, at least 30%, at least 40%, at least 50%, at least 60%, at least 70%, at least 80%, at least 90%, at least 95%, at least 98% post-mitotic neurons, and so on.

The starting population of neuronal progenitors is characterized by the presence of beta-III tubulin (Tuj1) positive cells, which could be mixed with other types of cells, for example astrocytes, fibroblasts, myoblasts or undifferentiated cells.

The culture is dissociated using a proteolytic enzyme such as trypsin for about 5-10 minutes, until the cells are rounded and detached from the substrate. The enzyme is washed from the cell suspension by repeated centrifugation and resuspension in media. Pre-existing cell agglomerates (or clumps) that are present as a result of incomplete dissociation are then removed by a gravitational method (sedimentation for 1 to 5 minutes or centrifugation at low g-force), or alternatively passed through a 40-100 micrometer cell strainer. The total number of cells is counted and then the cell suspension is placed onto a non-adherent surface, for example ultra-low adherent culture flask (Corning) at an optimal density empirically determined to be between about 100,000 to about 200,000 cells per cm².

In some aspects, the disclosure excludes a preparation of cells with a density less than 100,000 cells per cm², less than 75,000, less than 50,000, less than 25,000, less than 10,000, less than 5,000 cells per cm², and the like. In other aspects, the disclosure excludes a preparation of cells with a density greater than 200,000 cells per cm², greater than 300,000, greater than 400,000, greater than 500,000, greater than 750,000, greater than 1,000,000 cells per cm², and the like. What is also encompassed is any combination of less than and greater than densities, that is, what is encompassed are ranges of densities.

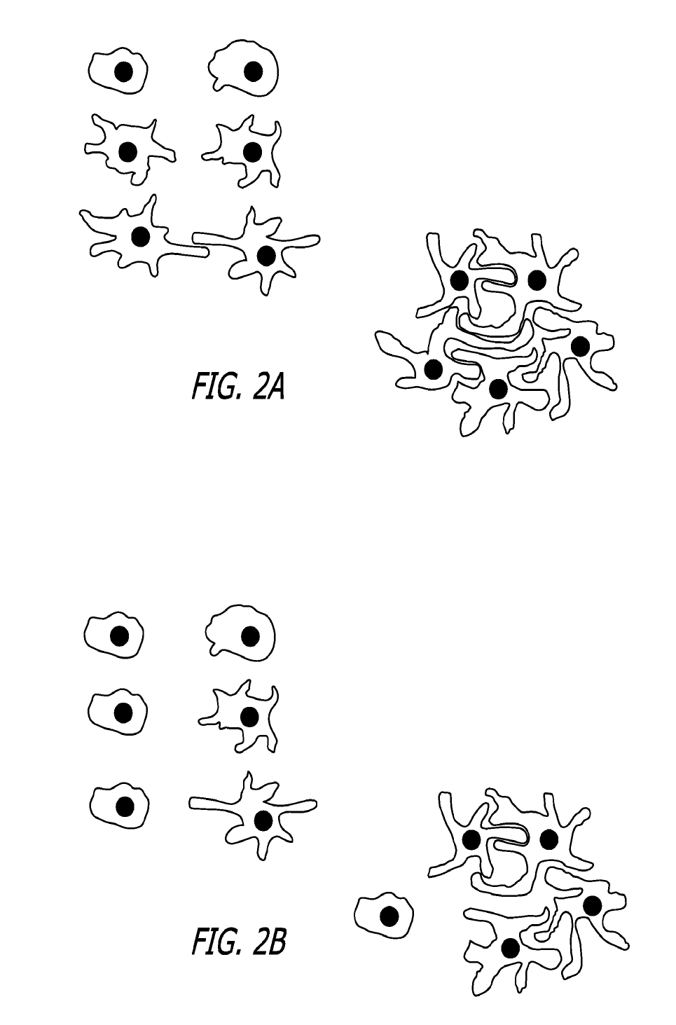

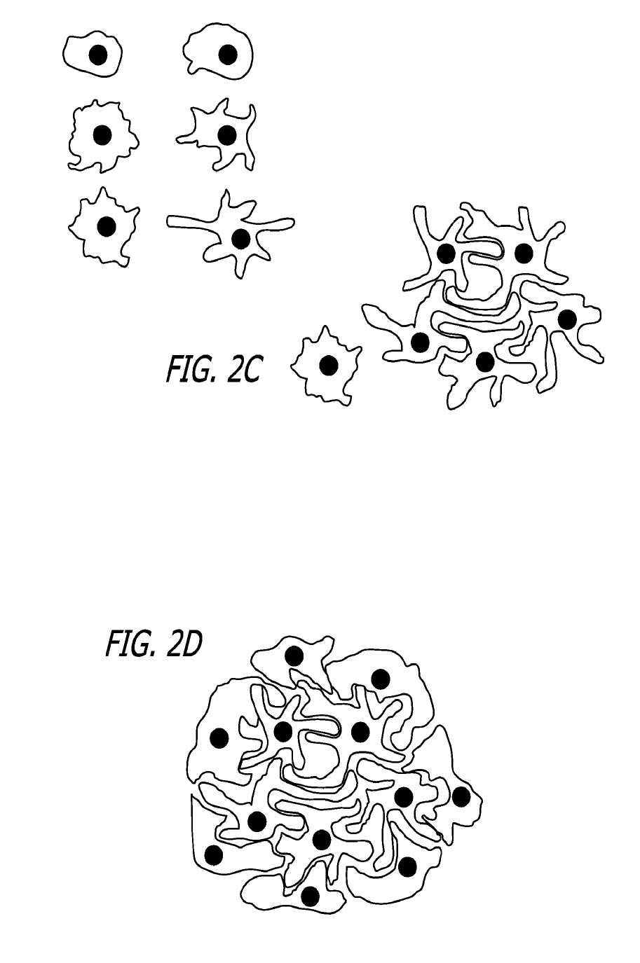

FIG. 2 discloses various interactions between live neural cells and other cells. FIG. 2A shows interaction of live neural cells, that is, cell to cell interaction during microsphere formation, showing: (1) Dissociated cells; (2) Exploratory cytoplasm expansion; (3) First contact, known as “handshaking;” and (4) Aggregation. FIG. 2B discloses interaction of live neural cell with a dead cell. Cell to cell interaction during microsphere formation, showing: (1) Dissociated cells; (2) Exploratory cytoplasm expansion only on the live cells; (3) No contact with the dead cell; and (4) Aggregation only of live cells. FIG. 2C reveals interaction of live neural cell with non-neural cell. Cell to cell interaction during microsphere formation, showing; (1) Dissociated cells; (2) Short exploratory cytoplasm expansion on non-neural cells; (3) Expansion not long enough to make contact; and (4) Aggregation only of neural cells. FIG. 2D illustrates cell to cell interaction during microsphere formation, showing that, after neural aggregation the cells on the periphery will send processes towards the inside of the sphere, preventing future recruitment from a distance.

A lower density will prevent formation of the microspheres, and a higher density will create spheres too large in size, or will incorporate non-viable or non-specific cells.

The cell suspension is placed to rest between 6 and 20 hours in a vibration free environment and with no disturbance during this time. The diagrams in FIGS. 2A-2D, represent the process of cell to cell adhesion and microsphere formation. In some instances referred to as homophilic cell to cell adhesion. At the end of resting time, the flask will contain a mixture of microspheres and non-neuronal single cells and debris. The media containing the microspheres is collected and the spheres are separated by gravity, for example centrifuged at low g-force (80-150 rcf), or by filtration. The exact centrifugation speed must be adjusted to the equipment specifics, therefore a tested centrifuge is recommended. Heavier microspheres will collect at the bottom of the tube while the supernatant will contain individual cells and debris. The individual cells could be dead or different types. If a strainer is used, a small mesh is recommended, for example 10-40 micrometers to retain the larger clumps. The cells from discarded supernatants are counted and subtracted from the original total. The centrifugation and counting of the discarded supernatant can be repeated to improve the removal efficiency. At the end of the washes, very few or no single cells should be observed in the microsphere suspension.

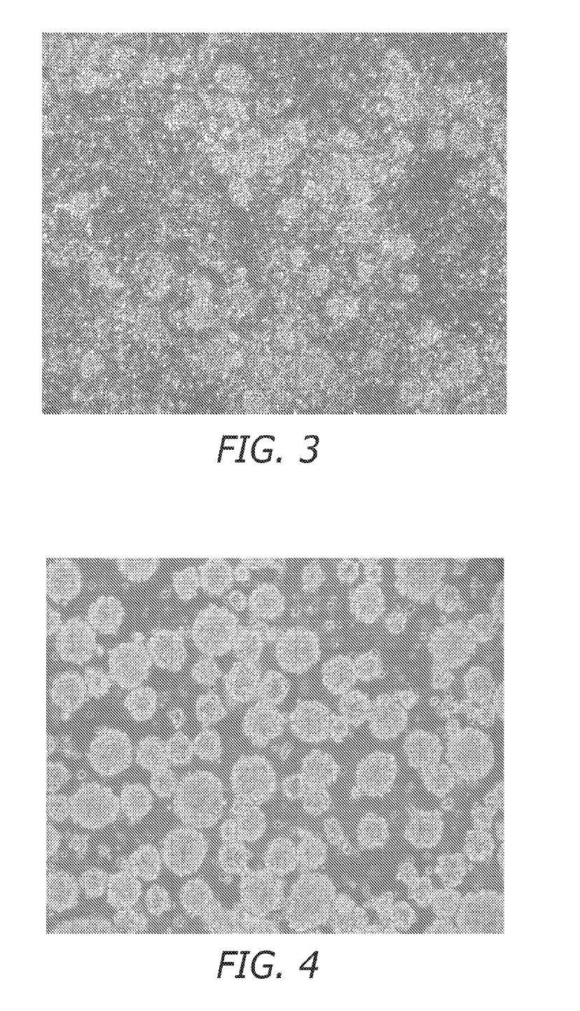

FIG. 3 is a photograph of microspheres after 12 hours of incubation, before wash (10×), while FIG. 4 shows microspheres after wash and concentration (20×). In FIG. 3, the undisturbed incubation resulted in relative homogenous size of spheres containing the neuronal cells. These spheres are surrounded by much debris and single cells. This debris is a typical result of an enzymatic dissociation procedure. The live single cells which are not incorporated in the microspheres are not viable or of a different type, for example fibroblasts and undifferentiated cells. In FIG. 4, which shows microspheres after wash, after first wash, the majority of debris is eliminated and the microspheres containing exclusive live neuronal cells, which can be harvested in high purity. A subsequent wash will eliminate the few single cells still floating around the microspheres.

In one aspect, the preparation of microspheres contains less than 10,000 observable free cells or single cells per microsphere, less than 5,000, less than 1,000, less than 500, less than 200, less than 100, less than 50, less than 10, less than 5, less than 2, and so on, observable free cells or single cells per microsphere.

### Device Used to Make Microspheres

A flat bottom cell culture vessel can be used for the microsphere process. This vessel must have a low adherent coating, for example agar or any other commercially available hydrophobic coatings, including commercially available products, for example ultra-low binding polystyrene cell culture flasks from Corning Life Sciences (Lowell, Mass.). Low adherent or non-adherent surfaces are described (see, e.g., Balasubramanian et al (2001) ASAIO 47:354-360; Kao et al (1994) J. Biomed. Mater. Res. 28:73-79, U.S. Pat. Nos. 6,365,405 issued to Salzmann et al, 4,248,685 issued to Beede et al, all of which are incorporated herein by reference).

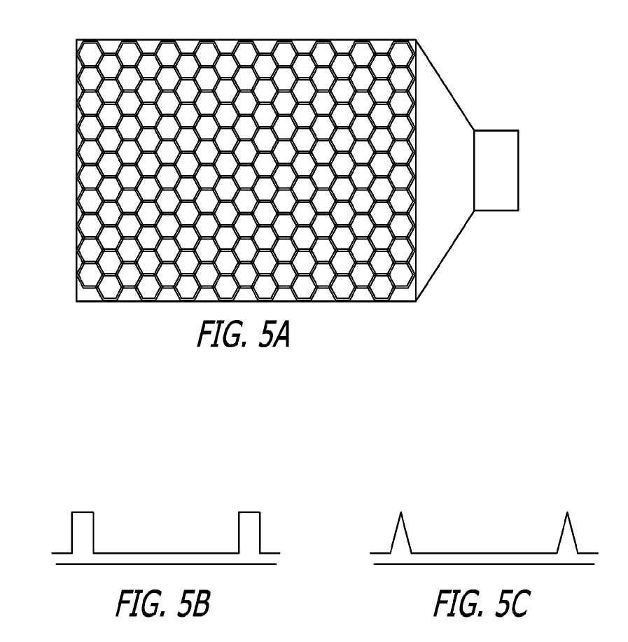

A preferred device is a cell culture flask depicted in FIG. 5, with the bottom extruded or etched to a 0.5 mm to 5 mm grid having raised walls about 0.01 to 0.1 mm could be used to obtain homogenous size of microspheres. Unlike previous systems to form cell agglomerates (embryoid bodies, neurospheres) with round bottom wells, this grid will have flat bottom wells, square or preferred hexagonal geometry for maximum surface occupancy and minimal corner effect. In a square geometry, the cells in the corner, given the greater distance to the center, could result in isolated smaller clusters, or left unincorporated into the microsphere. The round bottom wells cause the agglomeration of the cells in a very high density, or in a layered agglomeration; where a result is that microspheres may incorporate debris or nonviable cells. The flat bottom wells, with cells distributed on a single layer, encourage the active cell agglomeration, with reduced possibility to incorporate dead cells or non-neural cells. Hexagonal (honeycomb) wells are preferred, without implying any limitation, and square wells are suitable. It is preferred and recommended that the wells be flat on the bottom, not round, conical or pyramidal.

Another flat bottom microwell device was previously described using a stamped hydrogel matrix. The system described uses round wells of 100 micrometer diameter and 0.5 micrometer deep to capture single cells (Cordey et al, 2008).

In one aspect, the disclosure excludes devices and methods that use round bottom wells. In another aspect, the disclosure excludes devices and methods where wells cause or promote incorporation of debris or non-viable cells into microspheres.

### Software Used for Calculations

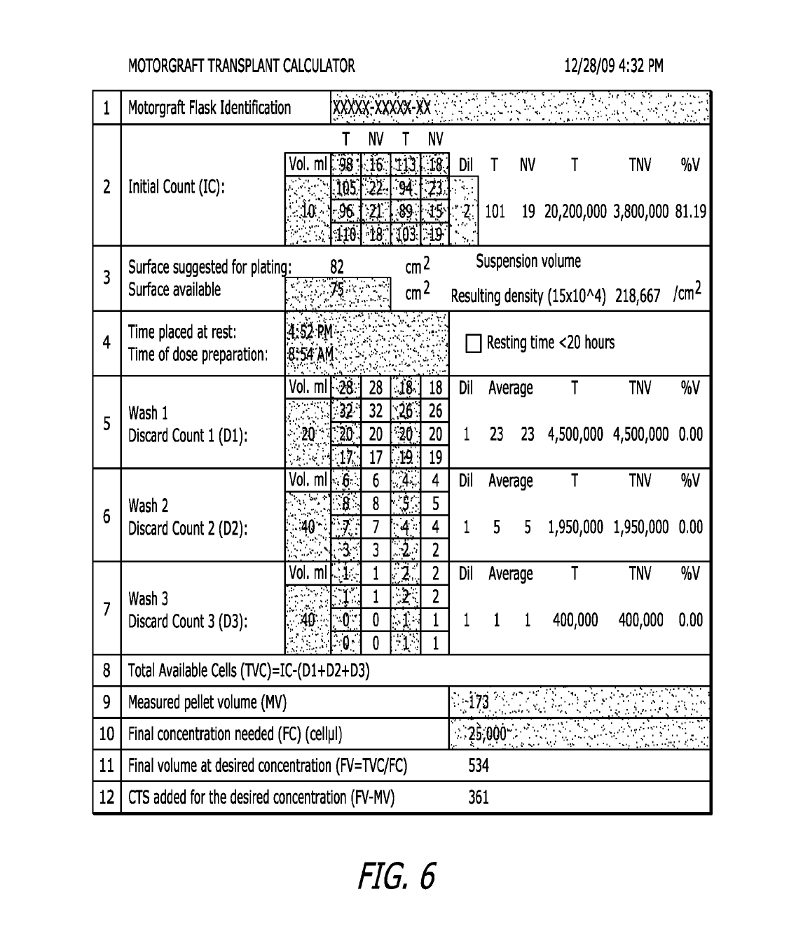

The actual number of cells contained in spheres is calculated by subtracting the number of cells counted in each supernatant discard during the low force centrifugation from the total count before resting. For the accuracy and speed of the calculations, an electronic form can be used. The form can use different levels of programming including but not limited to spreadsheets, e.g., Excel®, xml, Visual Basic®, C, optionally connected to database applications. The electronic form can be formatted to be used in good laboratory practice or good manufacturing practices environment. The electronic worksheet contains the calculations for manual cell counting, the required surface for resting, the calculations for manual debris counting, the dilutions required for transplant, viability and any calculations that are relevant to the process. An example of such worksheet is represented in FIG. 6. Similar data can be obtained with automated cell counters, for example, by flow-cytometry.

### EXAMPLES

### Example 1

### Cell Count Calculation Validation

This study evaluates two methods used for cell preparation for the purposes of transplantation. The traditional or classical enzymatic method (Method A) is compared to the new method (Method B), which separates viable cells from non-viable cells during the preparation of a high population of motor neuron progenitor cells, referred to herein as MotorGraft™, for transplantation based on the physiological properties of motor neuron progenitor (MNP) cells. Both methods were carried out and then viability and identity were examined.

### Cell Preparation

Method “A”—

In a traditional method (Method A) using dissociation by trypsin and viability evaluation by counting with Trypan Blue, counts are done immediately after cell preparation. Method A uses the cells immediately for counting, for example, within minutes

The cultures are dissociated using trypsin replacement (TrypLE) for 5 minutes at 37° C., 5 mL per 75 cm² flask. The dissociated cells were collected in a 50 mL tube and the volume in the tube is adjusted to 30 mL with HBSS (Hanks Balanced Salt Solution) or similar buffers, for example, Cell Transplant Solution (CTS), after the flask is rinsed.

Cells are centrifuged at 400 rcf for 3 minutes, the supernatant removed, and the pellet re-suspended in 30 mL of fresh CTS. The procedure of centrifugation is repeated and the pellet re-suspended in 30 mL of a neural media such as, for example NeuroBasal media (Life Technologies, Carlsbad, Calif.), DMEM:F12 with B27 or N2 supplement, or MotorBlast™ media. Ingredients of DMEM:F12, B27 supplement, N2 supplement, and other media, can be obtained from, for example, American Type Culture Collection (ATCC) (Manasses, Va.), Life Technologies (Carlsbad, Calif.), and Sigma Aldrich (St. Louis, Mo.). Ingredients of MotorBlast media: DMEM:F12 with B27 supplement, L-glutamine, insulin, sodium selenite, apotransferrin and magnesium chloride.

A 100 microliter cell suspension sample are taken from the middle of the cell suspension and mixed with 100 microliters Trypan Blue. Cell counts are performed using a hemocytometer. Counts are preformed from four 1 mm² squares from two counting chambers. Viable and nonviable cells are recorded separately.

Method B.

This method employs the microsphere formation; the counts are performed immediately after dissociation and after 12 hours after the procedure is completed. Method B involves: (1) An extended resting time of about 2 hours to 24 hours, typically 6 to 12 hours; and (2) Use of a non-adherent surface. It is recommended that the resting time not be longer than 24 hours. Beyond 22-24 hours, the cells inside the spheres start to organize and result in neurospheres. Also, beyond 22-24 hours, large agglomerates form which may impede the administration through a needle or catheter.

The cultures are dissociated by method “A” and the total number of cells including viable and non viable cells are counted and recorded (IC). The cells are interacted by placing (seeded) in low adherent 75 cm² cell culture flasks at a density of about 150,000 cells/cm², labeled with the original flask identification, and placed to rest in the CO₂ incubator overnight (digital image of culture are taken at the end of procedure). The preferred seeding density is about 150,000 cells/cm² (100,000 to about 200,000 cells per cm²).

Flasks are prepared by coating with 1:60 Matrigel™ (BD Biosciences, San Jose, Calif.), and kept overnight at 4° C.

The resting time, which should be 12 hours, are recorded without moving the flask. Microsphere size is recorded from at least 10 different microspheres at the beginning and the end of the resting time. Microsphere formation is examined and digital image of culture are taken.

The cell suspensions are collected in a 50 mL tube. The cells are centrifuged for 3 minutes at 200 rcf. The supernatant is collected and labeled “Discard 1” or “D1”. The volume is restored to 30 mL with HBSS or CTS and microsphere formation examined (Digital image of culture are taken). The cells are centrifuged for 3 minutes at 200 rcf. The supernatant is collected and labeled “Discard 2” or “D2”. The cells are resuspended again in HBSS or CTS, then centrifuged for 3 minutes at 200 rcf. The supernatant is collected and labeled “Discard 3” or “D3”. The pellets are re-suspended in 30 mL of MotorBlast™ and microspheres examined. Digital image of culture are taken.

Counts are performed from each discard using a hemacytometer, and the total particle number are calculated in each volume, by counting 4 mm squares in each chamber of the hemacytometer (total of 8 mm squares).

The total cell number in the microspheres is calculated by subtracting the cells in the discards (D1+D2+D3) from the total count that was performed on the previous day.

15 mL of the microspheres-containing suspension are then plated onto the Matrigel coated 4 well chamber slides. The other 15 mL of the microspheres are plated onto Matrigel coated T75 flask. On the following day, the supernatant are collected from the plated cells, and labeled Discard 4 (D4). A count are performed from D4 using a hemacytometer from 2×4 mm squares (total of 8 mm squares)

5 mL TrypLE are added to the adherent culture. The cells are incubated for 5 minutes. The cells are diluted with CTS to a volume of 30 mL. The cells are centrifuged at 250 rcf for 3 minutes. Supernatants are removed and the number of viable and nonviable cells from the pellet are counted.

Method “A” provides a percentage of viability which is recorded, which in the case of transplant population is corrected as transplanted volume to contain the desired viable cells.

Method “B” provides multiple data:

Total initial number of cells (IC).

Total Discarded Cells TD=D1+D2+D3

Total Viable Cells, TV=IC−TD

To verify the calculation of the total cells in the dose prepared by the method “B”, dissociation of the microspheres, followed by a count, should give a total very close to “TV” (total viable cells).

To verify the viability of the cells in the microspheres, the cells are allowed to attach (property only of live cells) and the floating cells are counted in the supernatant. To confirm again the total cells in the transplant dose, the adherent cells are dissociated and counted again to match closely the “TV”. The adherent cells were dissociated as follows. The cultures are dissociated using trypsin or trypsin replacement (TrypLE) for 5 minutes at 37° C., 5 mL per 75 cm² flask. The dissociated cells are motor neuron progenitor cells that were differentiated from human embryonic stem cells. The dissociated cells are collected in a 50 mL conical tube and the volume in the tube is adjusted to 30 mL with HBSS (Hanks' Balanced Salt Solution) or similar buffers. Cells are centrifuged at 400 rcf for 3 minutes, the supernatant removed, and the pellet re-suspended in 30 mL of fresh CTS. The procedure of centrifugation is repeated and the pellet re-suspended in 30 mL of media.

### Results

Three different batches of hESC line CSC14 were differentiated into MotorGraft™ cells and the microspheres were initiated on day 28 following the differentiation initiation. CSC14 is an embryonic stem cell line derived from blastocyst. Similar stem cell lines are H7 (WA07), H1 (WA01), and others. See, e.g., Nistor et al (2011) Derivation of high purity neuronal progenitors from human embryonic stem cells in PLos One 6:e20692.

For each batch, cell viability was determined for the starting population of cells (adherent MotorGraft™ cells) (IC) and the total viable cells (TVC) was also calculated from the cells retrieved after microsphere formation (Table 1).

A drop in viability of the initial count was expected, since additional cells will die during overnight incubation on non-adherent substrate. Washing steps were introduced to eliminate any damaged or dead cells and contaminant (other than motor neuron progenitors) cells not incorporated in microspheres (Table 2).

TABLE 1
Cell Viability before and after Microsphere Formation

| Batch lot number | IC | TVC | Drop in viability |
|---|---|---|---|
| MGRFT-DEC09- | 74.38% | 67.82% | 6.56% |
| 01 | | | |
| MGRFT-DEC09- | 91.38% | 85.37% | 6.01% |
| 04 | | | |
| MGRFT-JAN10- | 92.52% | 90.65% | 1.87% |
| 04 | | | |

TABLE 2
Cell Viability after Microsphere Plating

| | | Percentage viability of |
|---|---|---|
| | Percentage viability in | plated cells in flask |
| Batch lot number | growth media | following trypsinization |
| MGRFT-DEC09-01 | 99.999% | N/A |
| MGRFT-DEC09-04 | 99.996% | 99.976% |
| MGRFT-JAN10-04 | 99.998% | 99.997% |

### Example 2

### Lacing of MNPs with Human Embryonic Stem Cells

To test the efficiency to eliminate cells of different lineages, the MNPs were intentionally mixed with undifferentiated stem cells. Undifferentiated cells are particularly undesirable contaminants in any stem cell derived product intended for transplant because of the potential to form tumors after transplantation in living organisms. MNP cells were intentionally contaminated or “laced” with hESCs (6% and 20%), formed into microspheres, and then the discarded material from the washes and the microspheres were examined for expression of Oct-4, a marker for undifferentiated stem cells. Also, 100% hESCs were examined after the process of enzymatic dissociation and after the process of microsphere formation.

MNP cells were differentiated from hESCs according to a proprietary procedure. A flask of hESCs was also enzymatically dissociated and mixed with the MNP cells to result in a populations that contained undifferentiated hESC contaminants at 6% and 20%. The groups examined in this study are listed in Table 3.

TABLE 3
Groups Examined

| Group | % hESC | % MNP |
|---|---|---|
| 1 | 0 | 100 |
| 2 | 6 | 94 |
| 3 | 20 | 80 |
| 4 | 100 | 0 |

The following procedure, which is equivalent to the above-mentioned proprietary procedure, can be applied to any population of neural progenitors, neuronal progenitors and immature neurons obtained from embryonic parthenogenic, tumoral lines, or induced pluripotent stem cells. Primary cultures of neural or neuronal cells obtained from human tissue or other animal tissue can be subjected similarly to the purification procedure to eliminate cells introduced inadvertently during processing (fibroblasts, endothelial cells, smooth muscle cells, and the like). Methods to obtain differentiated neural or neuronal cells from the enumerated undifferentiated sources or from primary cultures are described in literature and can be practiced and implemented by the skilled artisan. See, for example, Dhara and Stice (2008) Neural Differentiation of Human Embryonic Stem Cells. J. Cell. Biochem. 105:633-640; Shin et al (2005) Human Motor Neuron Differentiation from Human Embryonic Stem Cells. Stem Cells and Development 14:1-4; Schulz et al (2003) Directed neuronal differentiation of human embryonic stem cells. BMC Neuroscience 4:27; Selvaraj et al (2012) Differentiating human stem cells into neurons and glial cells for neural repair. Front Biosci. 17:65-89; Isaev et al (2012) In vitro differentiation of human parthenogenetic stem cells into neural lineages. Regen Med. 7:37-45). In such instances the preferred method of purification, without implying any limitation, was differential attachment to substrates (for example cells non-adherent to uncoated plastic separated from the adherent cells by shaking, or rinsing the flask). Other methods use flow cytometery, also known as fluorescence activated cell sorting (FACS), to separate cells based on, for example, tagging with labeled antibodies. A surface coated with a specific antibody to a surface marker of a neuron can be used to selectively capture these cells by a method called immune-panning.

Cells were placed into a non-adherent flask at a density of 15,000 cells per cm² overnight, allowing the microspheres to form. The 100% hESC group, which serves as the positive control for Oct-4 immunostaining, was plated directly onto a Matrigel™ coated chamber slide. All cells were returned to the incubator overnight.

Following the above step, the entire contents of each flask were collected into 50 mL conical tubes and centrifuged at 150 rcf three times for 3 minutes to separate the microspheres from cells that did not incorporate into the microspheres. Each discard was counted for the discarded cells. The counts from the discards were used to calculate the total cells available after the washes. The available cells from the pellet were then resuspended and plated onto Matrigel™ coated chamber slides using and placed in the incubator overnight.

Discards were centrifuged at 250 rcf for 4 minutes. The supernatant was removed and the pelleted cells resuspended in media. The resuspended cells were plated onto Matrigel™ coated chamber slides.

Following overnight incubation of the plated cells, the cells were fixed with 4% paraformaldehyde for 10 minutes for immunostaining. The cells were then immunostained for Tuj1, a neuronal marker, and Oct-4, a marker of pluripotency. The Oct-4 antibody used recognizes the Oct-4A isoform, which is reported to be the only isoform of Oct-4 that plays a role in pluripotency. The detection threshold of immunocytochemical staining is sensitive enough to detect as low as 0.03% intentional hESC contaminants. Total number of cells, as determined by DAPI staining, was manually counted for each image using ImageJ software (National Institutes of Health, Bethesda, Md.). The total number of Oct-4 positive cells was also manually counted for each image and the percentage of Oct-4 positive cells was calculated. Tuj1 immunostaining demonstrated that Oct-4 negative cells were neuronal cells. Reagents and techniques for Oct-4 and Tuj1 immunostaining area available (see, e.g., US2009/0232779 of Keirstad and Nistor, which is hereby incorporated herein by reference).

A subset of cells from Group 1, 2, and 3 was plated onto Matrigel coated chamber slides prior to microsphere formation to analyze the percentage of Oct-4 positive cells before the microsphere formation process.

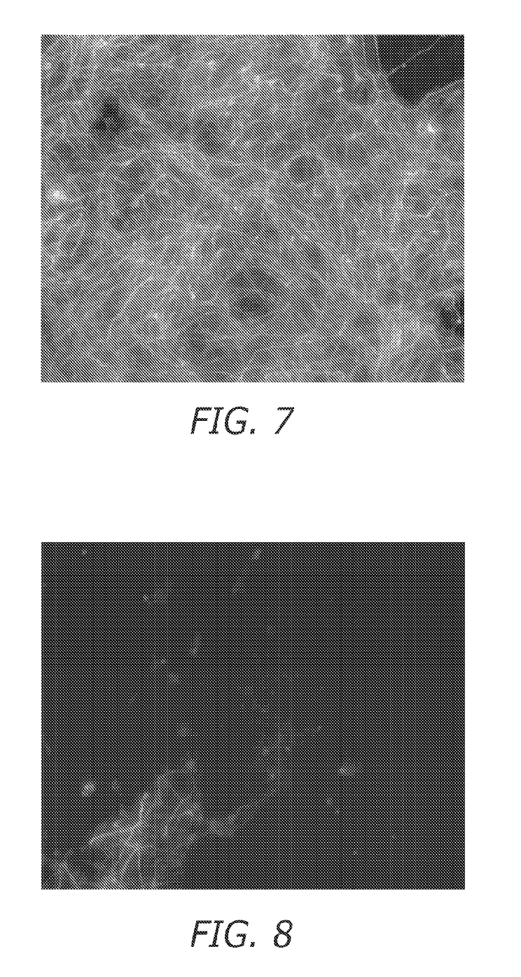

MNP cells that were not laced with hESC (Group 1) did not demonstrate expression of Oct-4 in the microspheres nor in the discarded material, as was expected (FIGS. 7, 8). There was extensive TUJ1 expression in the majority of the cells in Group 1 (FIG. 7).

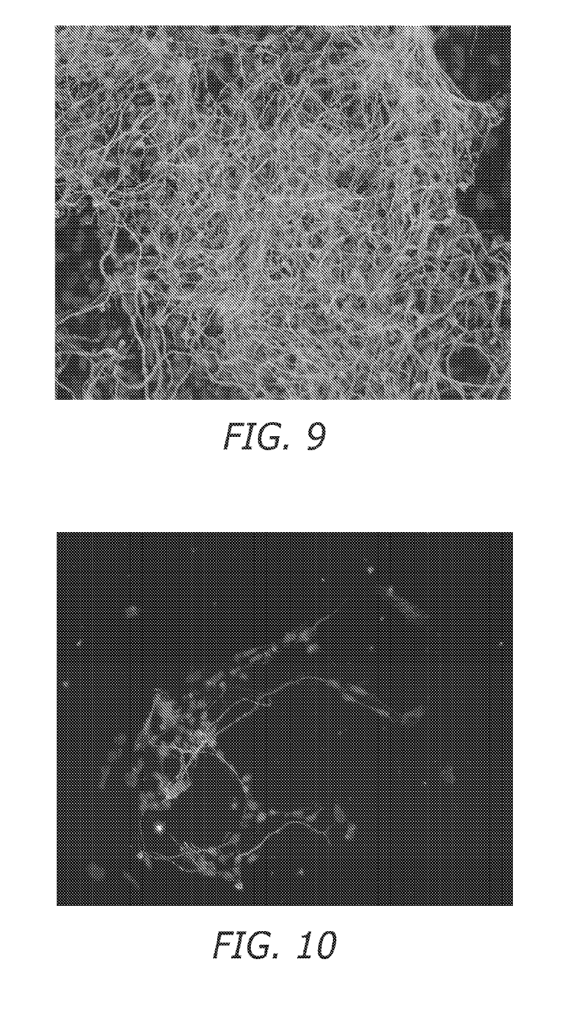

MNP cells that were intentionally laced with 6% hESC demonstrated few Oct-4 positive cells within TUJ1 positive cells. The majority of cells in the microspheres and the discards, however, were labeled with TUJ1 (FIGS. 9, 10).

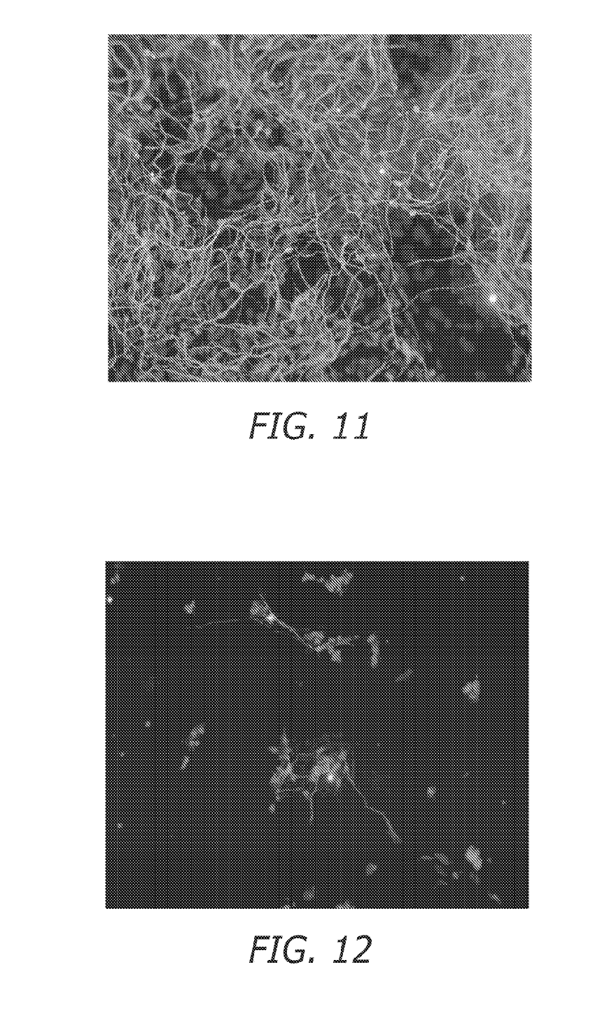

Similarly, MNP cells that were intentionally laced with 20% hESC demonstrated few, although qualitatively more than in the 6% hESC group, Oct-4 positive cells within TUJ1 positive cells (FIGS. 11, 12).

Cells that were 100% hESC and 0% MNP cells demonstrated extensive Oct-4 expression and no TUJ1 expression.

Prior to microsphere formation the number of undifferentiated hESC were lower than the intended lacing percentage (Table 4). There were 0% Oct-4 positive cells detected in the 0% hESC, 100% MNP population. There were 2.5% Oct-4 positive cells detected in the 6% hESC, 94% MNP population. There were 5% Oct-4 positive cells detected in the 20% hESC, 80% MNP population. There were 89.6% Oct-4 positive cells detected in the 100% hESC, 0% MNP population (Table 4).

TABLE 4
Percentage of Oct-4 positive cells before microsphere formation

| | Specified | |
|---|---|---|
| Group | Oct-4 | Actual observed Oct-4 |
| Group 1 (0% hESC, 100% MNP) | 0% | 0% |
| Group 2 (6% hESC, 94% MNP) | 6% | 2.5% |
| Group 3 (20% hESC, 80% MNP) | 20% | 5.0% |
| Group 4 (100% hESC, 0% MNP) | 100% | 89.6% |

Immunocytochemical staining of cells after microsphere formation demonstrates clearance of Oct-4 positive cell contaminants. MNP cells that were not laced with hESC (Group 1) did not demonstrate expression of Oct-4 after microsphere formation, as was expected. There was extensive TUJ1 expression in the majority of the cells in Group 1 following microsphere formation (FIG. 7).

Examination of the discard from Group 1 also demonstrated that there were no Oct-4 positive cells incorporated into the microspheres (FIG. 8). MNP cells that were intentionally laced with 6% hESC prior to microsphere formation (Group 2) demonstrated no Oct-4 positive cells within TUJ1 positive cells after microsphere formation, indicating that Oct-4 positive cells had been cleared from the population (FIG. 9).

Examination of the discarded material from Group 2 (6% hESC) also demonstrated that there were few or no Oct-4 positive cells left in the supernatant (FIG. 10).

MNP cells that were intentionally laced with 20% hESC prior to microsphere formation (Group 3) demonstrated few Oct-4 positive cells within TUJ1 positive cells after microsphere formation (FIG. 11).

Examination of the discarded material from Group 3 (20% hESC) also demonstrated that there were very few Oct-4 positive cells left in the supernatant (FIG. 11).

Quantitative assessment of Oct-4 positive cells after microsphere formation (Table 5) shows that no Oct-4 positive cells were detected in Group 2 (6% hESC). The discard from Group 2 contained an average of 0.1% Oct-4 positive cells. An average of 0.2% Oct-4 positive cells were detected in Group 3 (20% hESC) after microsphere formation. The discard from Group 3 contained an average of 0.1% Oct-4 positive cells.

TABLE 5
Percentage of Oct-4 positive cells after microsphere formation

| | | Observed | |
|---|---|---|---|
| | | Oct-4 after | Retrieved Oct-4 in |
| | Specified | microsphere | discard after |
| Group | Oct-4% | formation | microsphere formation |
| Group 1 | 0% | 0% | 0% |
| (0% hESC, 100% | | | |
| MNP) | | | |
| Group 2 | 6% | 0% | 0.1% |
| (6% hESC, 94% | | | |
| MNP) | | | |
| Group 3 | 20% | 0.2% | 0.1% |
| (20% hESC, 80% | | | |
| MNP) | | | |

The clearance of Oct-4 positive cells from MNP cells by one or more aspects of the herein disclosed system and/or process of microsphere formation was calculated by the following formula:

Clearance of Oct-4=((Oct-4 before microsphere formation−Oct-4 after microsphere formation)/Oct-4 before microsphere formation)*100

The calculations are as follows:

6% hESC, 94% MNPs:

2.480769%−0%=2.480769%

(2.480769%/2.480769%)*100=100% clearance

20% hESC, 80% MNPs:

5.01178%−0.174057%=4.837723%

(4.837723%/5.01178%)*100=96.52703% clearance

The herein disclosed microsphere methodology formation eliminated up to 100% of Oct-4 contaminants from MNP cells in the 6% hESC lacing group. Microsphere formation eliminates 96.5% of Oct-4 contaminants from MNP cells in the 20% hESC lacing group. The disclosed method can yield pure populations with of up to 100% elimination of Oct-4 contaminants.

### Example 3

### Process Applied to Non-Neural Type of Cells

In a subsequent experiment, undifferentiated populations hESCs alone were analyzed after the process of microsphere formation. hESCs were expanded and grown to confluency in five (5) T150 flasks. Once confluent, hESCs were enzymatically dissociated with TrypLE, as is done for microsphere formation with MNP cells. The dissociated cells were collected and rinsed, filtered through a 100 micrometer nylon mesh to eliminate cell clumps. The cell suspension was sampled (100 microliters) and combined with Trypan blue for counts of total and non-viable cells using a hemocytometer.

Half of the total cells were then placed into five (5) low adherent T75 flasks and were incubated 16 to 20 hours, to allow for microsphere formation to occur. The ‘Time placed at rest’ was recorded in the ‘MotorGraft Transplant Calculator’ Excel® sheet.

The remainder of cells were aliquoted into the six samples for Group A (see Table 6 for Group and Sample details).

TABLE 6
Groups and Samples

| | Day 1: | Day 2: Supernatant/ | |
|---|---|---|---|
| | Immediately | Debris of | Day 2: After cells |
| | after | suspended/sphered | suspended/sphered: |
| Group | dissociation ‘A’ | cells ‘B’ | “C” |
| 1 | PI, Oct-4, and Sytox | PI, Oct-4, and Sytox | PI, Oct-4, and Sytox |
| | Green: All colors | Green: All colors | Green: All colors |
| 2 | PI only | PI only | PI only |
| 3 | Oct-4 only | Oct-4 only | Oct-4 only |
| 4 | Sytox Green only | Sytox Green only | Sytox Green only |
| 5 | No primary Ab | No primary Ab | No primary Ab |
| | control (700) | control (700) | control (700) |
| 6 | No label | No label | No label |

Samples A1 and A2 were then labeled with PI. A concentrated PI solution was made by mixing 1225 microliters of PBS and 25 microliters of PI. The concentrated PI solution was added to the cell suspension at a 1:10 ratio of PI solution to cell suspension volume. The cells were incubated with PI for 30 minutes. Following incubation, the cells were washed to remove the PI solution, then fixed with a solution of 1% paraformaldehyde for 10 minutes. Following fixation, cells were washed then stored at 4° C. for immunocytochemical staining in PBS.

Samples A3-A6 were not labeled with PI, thus, they were fixed with a solution of 1% paraformaldehyde. Following 10 minute fixation, cells were washed then stored at 4° C. for immunocytochemical staining in PBS.

The cells that were set to rest for microsphere formation were removed from the incubator 17 hours after the ‘time placed at rest.’ The ‘time of dose preparation’ was recorded in the ‘MotorGraft Transplant Calculator’ Excel sheet. The contents of the flasks were collected, and then centrifuged, the supernatant was collected, and a 100 microliter sample was mixed with trypan blue for total and non-viable cell counts using a hemocytometer. The centrifugation procedure followed by cell count in the supernatant was repeated two more times and the cell count values were recorded for Wash 1 (Discard 1), Wash 2 (Discard 2) and Wash 3 (Discard 3) in the ‘MotorGraft Transplant Calculator’ Excel sheet.

The supernatant collected during the washes was pooled, centrifuged to pellet all cells and debris, then resuspended to be aliquoted into the six samples for Group B.

Samples B1, B2, C1, and C2 were then labeled with PI. A concentrated PI solution was made by mixing 1225 microliter of PBS and 25 microliter of PI. The concentrated PI solution was added to the cell suspension at a 1:10 ratio of PI solution to cell suspension volume. The cells were incubated with PI for 30 minutes. Following incubation, the cells were washed to remove the PI solution, and then fixed with a solution of 1% paraformaldehyde for 10 minutes. Following fixation, cells were washed then stored at 4° C. for immunocytochemical staining in PBS.

Samples B3-B6, and C3-C6 were not labeled with PI, thus, they were fixed with a solution of 1% paraformaldehyde for 10 minute fixation, washed then stored at 4° C. for immunocytochemical staining in PBS.

Samples A1, A3, A5, B1, B3, B5, C1, C3, and C5 were processed for immunostaining simultaneously. Samples were centrifuged at 1800 rpm for 3 minutes, then resuspended in 0.1% Triton X-100 for 15 minutes. Samples were then centrifuged again and resuspended for overnight incubation at 4° C. in:

A1, B1, C1, A3, B3, C3: 1:500 dilution of polyclonal rabbit anti-Oct-4 primary antibody (Santa Cruz, S.C.-9081/(H-134)), diluted in 0.1% BSA in PBS

A5, B5, C5: 0.1% BSA in PBS

Following overnight incubation, samples were centrifuged at 1800 rpm for 3 minutes. Samples were washed twice with PBS. Samples were then resuspended for 30 minute incubation at room temperature in:

A1, B1, C1: 1:2000 dilution of Alexa Fluor 700 goat anti-rabbit secondary antibody and 1:5000 dilution of Sytox Green® (Molecular Probes, Eugene, Oreg.) in PBS

A3, B3, C3, A5, B5, C5: 1:2000 dilution of Alexa Fluor 700 goat anti-rabbit secondary antibody (Life Technologies, Carlsbad, Calif.) in PBS

Samples A4, B4, and C4 will then be centrifuged at 1800 rpm for 3 minutes and resuspended for 30 minute incubation at room temperature in 1:5000 dilution of Sytox Green in PBS.

All samples that were incubated in secondary solutions were then centrifuged at 1800 rpm for 3 minutes, washed one time in PBS, centrifuged again, then resuspended in 500 μL of PBS. Samples were stored at 4° C. until further analysis.

All samples were analyzed for Oct-4, PI, and Sytox Green for nuclear counterstain. Only cells with a positive nuclear stain were included in statistics.

### Results

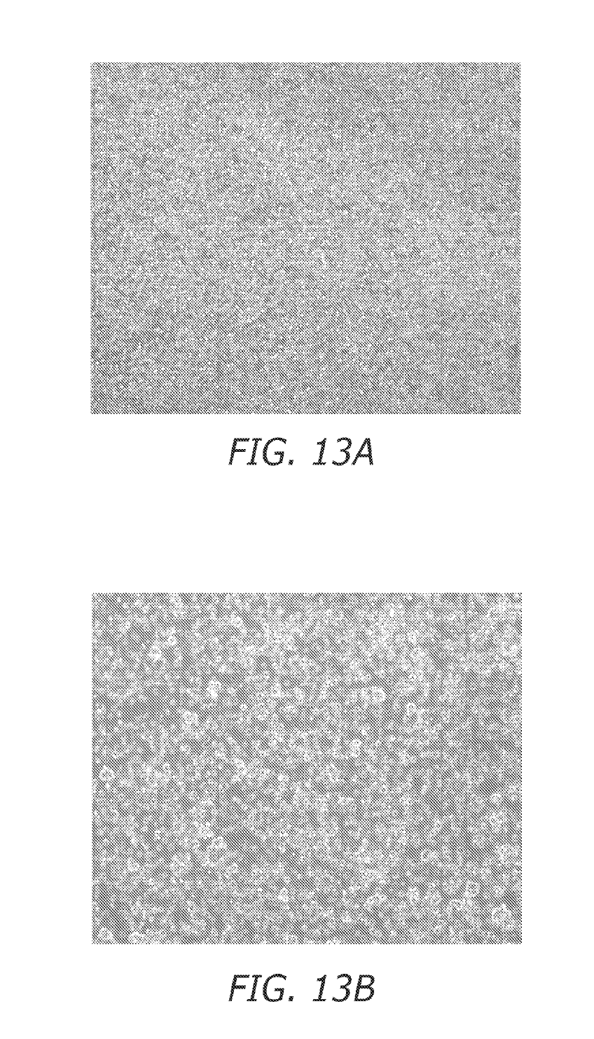

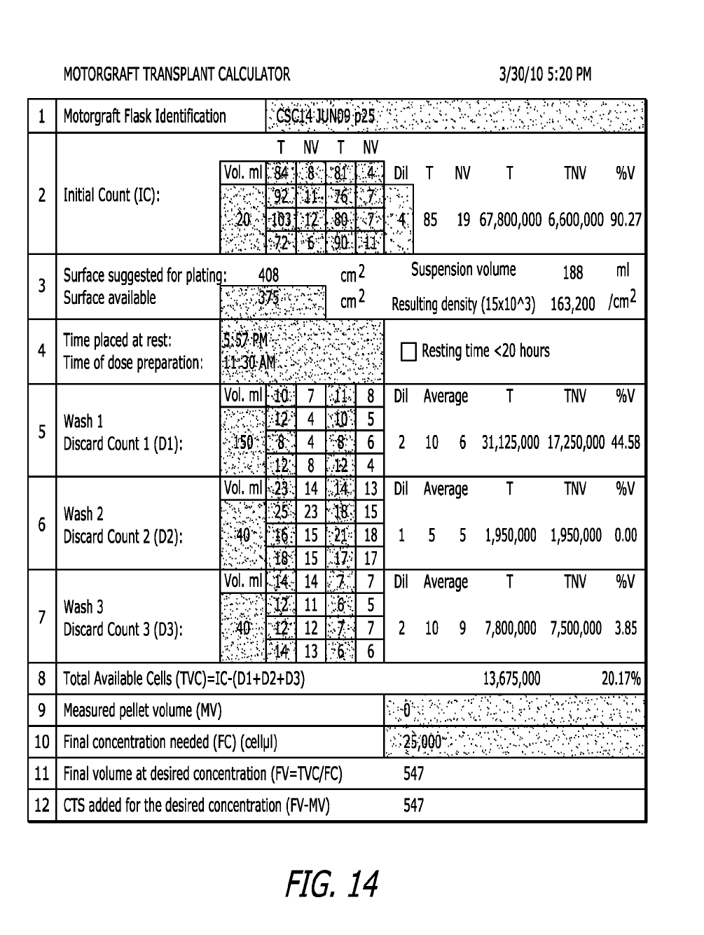

After 17 hours at rest, hESCs did not form microspheres, very few clumps were observed resembling small embryoid bodies (FIG. 13). The initial hESC count before microsphere formation was 135,600,000. Half of these hESCs, 67,800,000 cells, underwent the microsphere formation process. From the original 67,800,000 cells placed in the flask to form microspheres, there were a total of 13,675,000 viable hESCs available after the microsphere formation process. Thus, there was a 79.8% drop in the hESCs during microsphere formation. Only about 20% of the hESCs remain viable following microsphere formation. See the cell count worksheet of FIG. 14.

After overnight resting, only 0.6% of the cells in the clumps were Oct4 positive and viable, 1.7% Oct4 positive and nonviable, while the rest of the cells were Oct4 negative viable (41.7%) or nonviable (56%)

This experiment demonstrates that a population consisting non-neural cells, exemplified by using the undifferentiated stem cell cultures, do not efficiently form cell agglomerates, resulting mostly in cell death after dissociation. Overnight incubation of these non-neural cells in non-adherent conditions results in 20.7% viability, and from the surviving cells, only 0.6% being undifferentiated stem cells. Thus this method obtains only a 0.12% survival of undifferentiated stem cells through the process of microsphere formation. This confirms the previous lacing experiment, when 20% undifferentiated stem cells were mixed with MNP and only about 0.2% of Oct4 positive cells survived after microsphere formation.

The following parameters are contemplated. In one aspect, contaminating cells account for less than 30% of total cells, less than 25%, less than 20%, less than 15%, less than 10%, under 8%, less than 6%, less than 4%, and the like, of total cells. In another aspect, cells that are non-neural cells account for less than 30% of total cells, less than 25%, less than 20%, less than 15%, less than 10%, under 8%, less than 6%, less than 4%, and the like, of total cells.

The disclosure provides a population of microspheres (or of a given population of cells), where at least 20% of the cells detectably express Tuj1, where at least 30%, at least 40%, at least 50%, at least 60%, at least 70%, at least 80%, at least 85%, at least 90%, at least 95%, or at least 98% of the cells detectably express Tuj1. In other embodiments, what is excluded is a population of microspheres (or of a given population of cells), where less than 90% of the cells detectably express Tuj1, where less than 85%, less than 80%, less than 70%, less than 60%, less than 50%, and so on, detectably express Tuj1. Without implying any limitation, expression can be detected by antibodies, antibody-derived binding compounds, nucleic acid hybridization techniques, and the like.

The disclosure, in some embodiments, provides a method to reduce contamination in a cell population, wherein cells are incubated in the culture medium for a predetermined period of time (or a resting time), and wherein the period of time is less than 84 hours, less than 72 h, less than 60 h, less than 36 h, less than 30 h, less than 27 h, less than 24 h, less than 21 h, less than 18 h, and the like, or wherein the period of time is sufficiently short to avoid formation of large agglomerates that may impede administration through a needle or through a catheter. The period of time can be, without limitation, 6-12 h, 6-15 h, 6-18 h, 6-21 h, 6-24 h, 6-27 h, 6-30 h, 8-12 h, 8-15 h, 8-18 h, 8-21 h, 8-24 h, 8-27 h, 8-30 h, 10-12 h, 10-15 h, 10-18 h, 10-21 h, 10-24 h, 10-27 h, 10-30 h, 12-15 h, 12-18 h, 12-21 h, 12-24 h, 12-27 h, 12-30 h, 15-18 h, 15-21 h, 15-24 h, 15-27 h, 15-30 h, and the like. Also provided, is a population of cells prepared by said method, as well as a purified population of cells prepared by said method.

The disclosure provides a population of microspheres (or of a given population of cells), wherein less than 80% of the cells detectably express Oct-1, wherein less than 70%, less than 60%, less than 50%, less than 40%, less than 30%, less than 25%, less than 20%, less than 15%, less than 10%, less than 5%, less than 2%, and the like, detectably express Oct-1.

### REFERENCES

Reynolds B A, Rietze R L. Neural stem cells and neurospheres—re-evaluating the relationship. Nat. Methods. 2005 May; 2(5):333-6.
B A Reynolds and S Weiss. Generation of neurons and astrocytes from isolated cells of the adult mammalian central nervous system. Science 27 Mar. 1992. Vol. 255 no. 5052 pp. 1707-1710
Mori H, Fujitani T, Kanemura Y, Kino-Oka M, Taya M. Observational examination of aggregation and migration during early phase of neurosphere culture of mouse neural stem cells. Biosci Bioeng. 2007 September; 104(3):231-4.
Mori H, Ninomiya K, Kino-Oka M, Shofuda T, Islam M O, Yamasaki M, Okano H, Taya M, Kanemura Y. Effect of neurosphere size on the growth rate of human neural stem/progenitor cells. J Neurosci Res. 2006 December; 84(8):1682-91.
Jeffrey C Mohr, Juan J de Pablo and Sean P Palecek. 3-D microwell culture of human embryonic stem cells. Biomater 27(36):6032-42 (2006)
Karp J M, Yeh J, Eng G, Fukuda J, Blumling J, Suh K Y, Cheng J, Mandavi A, Borenstein J, Langer R, Khademhosseini A. Controlling size, shape and homogeneity of embryoid bodies using poly(ethylene glycol) microwells. Lab Chip. 2007 June; 7(6):786-94. Epub 2007 May 2.
Loic P. Deleyrolle, Rodney L. Rietze, Brent A. Reynolds. The neurosphere assay, a method under scrutiny. Acta Neuropsychiatrica 2008:20:2-8
JENSEN J B, PARMAR M. Strengths and limitations of the neurosphere culture system. Mol Neurobiol 2006; 34:153-161.
Tian L, Catt J W, O'Neill C, King N J. Expression of immunoglobulin superfamily cell adhesion molecules on murine embryonic stem cells. Biol Reprod. 1997 September; 57(3):561-8.
Myriam Cordey, Monika Limacher, Stefan Kobel, Verdon Taylor, Matthias P. Lutolf. Enhancing the Reliability and Throughput of Neurosphere Culture on Hydrogel Microwell Arrays. STEM CELLS, Volume 26, Issue 10, p. 2586-2594, October 2008

Although the present invention has been described in connection with the preferred form of practicing it, those of ordinary skill in the art will understand that many modifications can be made thereto without departing from the spirit of the present invention. Accordingly, it is not intended that the scope of the invention in any way be limited by the above description.

1. A method to reduce contamination in a cell population, the method comprising:
incubating a suspended population of live cells in a culture medium;
with a substrate having surface that promotes cell to cell adhesion;
wherein the incubating is in absence of mechanical disturbance of the culture medium;
wherein cells are incubated in the culture medium for a predetermined period of time;
resulting in a population of cells that is relatively reduced or eliminated in contaminants; and,
wherein the population of cells that is relatively reduced or eliminated in contaminants is collectable by centrifugation and is separable from said contaminants by centrifugation.

2. The method of claim 1, wherein the substrate is a low adherent coating.

3. The method of claim 1, wherein the substrate is a low adherent coating that comprises one or more of a highly hydrophobic material, fluoropolymer, polyethylene, or polystyrene.

4. The method of claim 1 wherein containments which are reduced or eliminated are at least one of non-viable cells and undifferentiated stem cells.

5. The method of claim 1 wherein the substrate has surface properties that encourage or enhance cell to cell adhesion.

6. The method of claim 1 wherein the population of live cells are neural cells.

7. The method of claim 1 wherein the population of live cells are neuronal progenitors.

8. The method of claim 1, wherein the substrate has a bottom that is substantially flat, wherein the flat bottom minimizes cell agglomerates, as compared to a substrate that comprises a round bottom well.

9. The method of claim 1 wherein the population of live cells which are incubated or interacted with the substrate are seeded at a density of about 100,000 to about 200,000 per cm².

10. The method of claim 1 wherein the predetermined time is between about 3 and about 24 hours.

11. The method of claim 9, the method further comprising keeping the cell suspension generally undisturbed by manipulations, in a substantially vibration free environment.

12. The method of claim 8 the method further comprising prior to seeding removing pre-existing cell agglomerates by a gravitational method.

13. The method of claim 10, wherein the medium contains microspheres, and the wherein the microspheres are collected by a gravitational method or by filtration.

14. The method of claim 12, wherein the gravitational method is centrifugation at low gravity force, and wherein the low gravity force is 80-150 relative centrifugal force (ref).

15. The method of claim 11, wherein the medium contains microspheres, and the wherein the microspheres are collected by a gravitational method or by filtration.

16. A cell population prepared by the method of claim 1, wherein the cell population is derived from human motor neuron progenitor cells, wherein at least 50% of the cells detectably express Tuj1, wherein fewer than 10% of the cells detectably express Oct-4, and wherein at least 50% of the cells reside in at least one microsphere.

17. The cell population of claim 14, wherein at least 80% of the cells detectably express Tuj1.

18. A device to form consistent size and composition microspheres comprising: a substantially flat bottom container; and,
a low adherent substrate coated on the inner surface of said container.

19. The device of claim 18 wherein said low adherent substrate comprises at least one of polystyrene, polyethylene, and fluoropolymers.

20. The device of claim 17 wherein said low adherent substrate comprises a low adherent coating.

21. The device of claim 20 wherein said low adherent coating is applied via at least one of extruding, stamping, photo resist, and as a film.

25. (canceled)

26. (canceled)

22. The device of claim 18 wherein the container further comprises wells.

23. The device of claim 22 wherein the wells are between about 0.01 mm to about 0.1 mm deep.

24. The device of claim 22 wherein the wells are substantially polygonal in shape.
